(19) United States
(12) Patent Application Publication
(10) Pub. No.: US 2014/0228645 A1
(43) Pub. Date: Aug. 14, 2014

(54) LARYNGOSCOPE BLADE

(71) Applicant: Idean Ahmad Pourshams, San Jose, CA (US)

(72) Inventor: Idean Ahmad Pourshams, San Jose, CA (US)

(73) Assignee: Idean Ahmad Pourshams, San Jose, CA (US)

(21) Appl. No.: 14/179,528

(22) Filed: Feb. 12, 2014

Related U.S. Application Data
(60) Provisional application No. 61/764,323, filed on Feb. 13, 2013.

(51) Int. Cl. A61B 1/267

(52) U.S. Cl. CPC A61B 1/267
USPC 600/190

(57) ABSTRACT

In one embodiment, the present application discloses a laryngoscope blade assembly comprising a blade body and an elastomeric material configured on the blade body, wherein the elastomeric material is permanently adhered to the lower surface, the upper surface or both the lower and the upper surfaces.

### BACKGROUND OF THE INVENTION

Laryngoscopes are devices widely used in surgical practice and emergency settings. Laryngoscopes commonly comprise a handle and a blade portion. A laryngoscope blade may be used to displace a patient's tongue and epiglottis for better visualization of the larynx. A laryngoscope blade also assists to ease the placement of an endotracheal tube, i.e. endotracheal intubation, mechanical ventilation, and the delivery of anesthetic agents during surgical procedures. Endotracheal intubation is a common medical method to ensure that medical personnel has access to and control of an unobstructed airway to deliver oxygen, and other therapeutic agents into patients' lungs and trachea. During the intubation, it is common that substantial maxillary and mandibular injury results when the laryngoscope blade collides with the patient's jaws, teeth or gums, throat and pharynx with a sufficiently strong force. The intubation procedure requires that a patient lie on his back. A user inserts the blade into a patient's mouth, with the tip of the laryngoscope blade entering first. The free end of the handle which is not attached to the blade points away from the face of the patient. The user then grasps and pulls the handle in the same direction that the free end of the handle points to. The pulling is done with a sufficiently strong force to open up the airway. During such procedure that is often performed under emergency situation, the medical personnel may inadvertently use the patient's jaw, teeth, or gums as a point of leverage to open up the airway, i.e. “rocking the blade”. As a consequence of such procedure, the patient may suffer dental damage resulting in chipped and broken teeth or dentures, or where the teeth or dentures may be dislodged from the jaw. Significantly, dislodged teeth or dentures may fall into the patient's airway, creating obstruction or resulting in further injury. Additionally, the gums as well as the maxillary and mandibular alveolar process are usually contused or torn. Such damage that may be inflicted in a patient wearing dentures may further cause deformation of the surrounding soft and hard tissues such that the patient's dentures may no longer be securably attached since the surrounding soft and hard tissues are deformed. The resulting damage inflicted on a patient may also damage the periodontal ligament that holds the teeth.

Laryngoscope blades with a disposable or detachable cover designed to reduce injury have certain disadvantages, including potential fluid cross contamination from a patient. There may be potential loss of crucial time in manually attaching the cover to a blade in an emergency situation. It may also be cumbersome to manually attach and detach the cover for cleaning and sterilization of the blade and cover. In addition, such contact points or joints built onto the blade may collect bacteria or food particles at points of contact between the cover and the blade.

In addition, commercially available blades with or without such protective cover often suffer from undesirable and sudden movement or slippage when deployed in a patient's mouth. That is, when the blade is inserted and positioned into a patient's mouth and the blade is rested and placed in contact with one or more location on the wet and slippery oral mucosa, the blade may suddenly slip and be displaced from the desired position, causing injury to a patient's mouth.

Lastly, emergency airways management with rapid sequence intubation requires crucially precise and error free procedures. The clinical situations often involve blood, vomited matter, secretions and potentially injured or distorted anatomy with rapidly deteriorating patients. Laryngoscopy with rapid and precise intubation sequence is challenging in this situation, but speed and accuracy are crucial. If a blade cannot be securely positioned while a patient is suffering from impaired breathing or obstructed airway, the medical personnel may have a very difficult time placing the endotracheal tube.

Accordingly, there is an unmet need for a laryngoscope blade that does not cause dental, maxillary or mandibular injury to the patient. There is also an unmet need for a blade that may be operated expeditiously without a risk for cross-contamination of biological matter. There is also an unmet need for a blade that may be employed safely and eliminate or prevent inadvertent slippage when inserting into the patient's mouth, preventing injury to the oral mucosa, the epiglottis and the airway. In addition, there is also an unmet need for a blade that may provide secured displacement of the tongue and better illumination of the mouth and the airway, resulting in better visualization of the trachea during an intubation procedure.

### SUMMARY OF THE INVENTION

In one aspect of the present application, there is provided a laryngoscope blade assembly that may be used to significantly reduce or eliminate the risk of dental, maxillary or mandibular injury to the patient. In another aspect of the present application, there is provided a laryngoscope blade assembly that may be used and re-used without a risk for cross-contamination, wherein the blade assembly is configured such that it eliminates the need for engaging, replacing or using a detachable or removable protective cover, or a protective dental guard mouthpiece which some anesthesiologists use to protect the teeth during surgery, saving the user significant time and effort in both emergency and elective situations.

In another aspect of the present application, there is provided a laryngoscope blade assembly that may reduce the risk, eliminate or prevent the inadvertent slippage of the blade when using and resting the blade in the patient's mouth, thus preventing oral mucosal, epiglottal and tracheal injury or contusions. In another aspect of the present application, there is provided a laryngoscope blade assembly, wherein the blade assembly may provide effective displacement and securing of the tongue during intubation, and provide better illumination of the mouth and the airway, resulting in significant improvement in visualization of the trachea. In one aspect of the present application, there is provided a laryngoscope blade assembly, wherein the blade assembly may comprise an improved light source, an improvement of the placement of the light source, and wherein the light source may include one or more lights. In another aspect of the present application, there is provided a laryngoscope blade assembly, the blade assembly comprises an elastomeric material to allow for an improved, more convenient, cost efficient and ease of use compared to other blades existing on the market. Furthermore, the blade of the present application is designed to prevent or eliminate injury to the teeth, gums, or jaw, inadvertent blade slippage in a patient's mouth, and fluid cross-contamination.

In yet another aspect of the present application, there is provided a laryngoscope blade assembly wherein the blade assembly may be adaptively configured to protect the patient's teeth, jaws, gums, tongue and oral mucosa. The blades may be any and all types of laryngoscope blades, whether the blades are straight, curved, partially curved, or curved distally, proximally, laterally or medially. For example, such blades include the Miller blade, the Macintosh blade or the Magill blade.

In one aspect of the present application, there is provided a laryngoscope blade assembly having a proximal end, a distal end and a distal tip, comprising a blade body comprising an upper face and a lower face, a proximal end and a distal end, the upper face and lower face defined by a first edge and a second edge; wherein the first edge of the blade body is configured to connect to a top edge of a substantially vertical portion of a flange having a proximal end and a distal end, wherein the flange comprises the vertical portion comprising the top edge and a bottom edge, a lateral side wall and a medial side wall, the wall defined by the top edge and the bottom edge, wherein the vertical portion extends downward from the first edge of the blade body, and the flange further comprising a substantially horizontal portion, wherein the horizontal portion comprises an upper surface and a lower surface; wherein the horizontal portion is configured to extend laterally or medially from the bottom edge of the vertical portion; and an elastomeric material configured on the lower surface of the horizontal portion of the flange, wherein the elastomeric material is permanently adhered to the lower surface or upper surface, or both lower and upper surfaces; wherein the blade body is substantially arcuate with the convex upper face and a concave lower face; and wherein the horizontal portion of the flange is substantially arcuate with the convex upper surface and a concave lower surface.

In one aspect, the horizontal portion is configured to extend laterally from the bottom edge of the vertical portion, in a direction substantially away from the blade body. In another aspect, the elastomeric material is configured on the lateral side wall of the vertical portion of the flange, wherein the material is permanently adhered to the lateral side wall. In one aspect, the elastomeric material comprises one or more layers. In another aspect, the elastomeric material is adaptively configured to enclose one or more air-tight cavities, wherein the cavity is adapted to enclose an air pocket. In one aspect, the elastomeric material is adaptively configured to incorporate one or more light sources, wherein the light source is configured on the vertical portion of the flange, the horizontal portion of the flange, the blade body, the distal end and proximal end of the blade assembly, the distal tip of the blade assembly, and combinations thereof.

In one aspect, the elastomeric material comprises an exterior surface and an interior surface, wherein the interior surface is configured to contact and permanently adhered to the blade assembly, wherein the exterior surface comprises a textured surface. Such textured surface may be selected from bumps, raised protrusions, knobs, riblets, waves, ridges, ribs, raised truncated domes or half domes, or combinations thereof. In one aspect, the elastomeric material and the lower surface of the horizontal portion of flange comprise a substantially continuous surface. In one aspect, the elastomeric material and the upper face of the blade body comprise a substantially continuous surface. In one aspect, the elastomeric material and the lateral side wall of the vertical portion of the flange comprise a substantially continuous surface. In one aspect, the blade is selected from the group consisting of a Macintosh, Miller or Magill blade.

In one aspect, there is provided a laryngoscope blade assembly having a proximal end, a distal end and a distal tip, comprising a blade body comprising an upper face and a lower face, a proximal end and a distal end, the upper face and lower face defined by a first edge and a second edge; wherein the first edge of the blade body is configured to connect to a top edge of a substantially vertical portion of a flange having a proximal end and a distal end, wherein the flange comprises the vertical portion comprising the top edge and a bottom edge, a lateral side wall and a medial side wall, the wall defined by the top edge and the bottom edge, wherein the vertical portion extends downward from the first edge of the blade body, and the flange further comprising a substantially horizontal portion, wherein the horizontal portion comprises an upper surface and a lower surface; wherein the horizontal portion is configured to extend medially from the bottom edge of the vertical portion, in substantially the same direction as the blade body; and an elastomeric material configured on the lower surface of the horizontal portion of the flange, wherein the elastomeric material is permanently adhered to the lower surface or upper surface, or both upper and lower surfaces; wherein the blade body is substantially arcuate with the convex upper face and a concave lower face; and wherein the horizontal portion of the flange is substantially arcuate with the convex upper surface and a concave lower surface.

In one aspect, the elastomeric material comprises a thickness from about 2 mm to about 10 mm, from about 4 mm to about 8 mm, and from about 5 mm to about 7 mm.

In one aspect, the laryngoscope blade can be constructed from plastic or metal alloy.

In another aspect, the laryngoscope blade may be used as a disposable blade. In one embodiment, the laryngoscope blade configured with an elastomeric material may be used as a disposable blade.

In one aspect, the laryngoscope blade assembly further comprises a mounting portion and a handle having a proximal end and a distal end, wherein the handle is pivotally and lockably attached at the proximal end to the mounting portion of the laryngoscope blade assembly. In one aspect, the handle is pivotally and lockably attached to the laryngoscope blade assembly. The pivoting and locking action of the handle with respect to the blade allows the user to adjust and lock the angle between the handle and the blade, which may be substantially at 90 degrees, or being at an acute or obtuse angle. In one embodiment, the handle and the laryngoscope blade assembly is constructed as a unitary construction, that is the handle and the blade assembly is formed from a single cast or mold. In one embodiment, the unitary-constructed handle and blade assembly is disposable.

In another aspect, the handle is adaptively configured with a power source and electrical conduit to one or more light sources configured on the blade assembly. In one aspect, the handle is adaptively configured with an internal light source and an optical conduit, wherein the internal light source is disposed within the handle.

In one aspect, there is provided a laryngoscope blade assembly having a proximal end, a distal end and a distal tip, comprising a blade body comprising an upper face and a lower face, a proximal end and a distal end, the upper face and lower face defined by a first edge and a second edge; wherein the first edge of the blade body is configured to connect to a top edge of a substantially vertical portion of a flange having a proximal end and a distal end, wherein the flange comprises the vertical portion comprising the top edge and a bottom edge, a lateral side wall and a medial side wall, the wall defined by the top edge and the bottom edge, wherein the vertical portion extends downward from the first edge of the blade body, and the flange further comprising a substantially horizontal portion, wherein the horizontal portion comprises an upper surface and a lower surface; wherein the horizontal portion is configured to extend medially from the bottom edge of the vertical portion; and an elastomeric material configured on the lower surface of the horizontal portion of the flange, wherein the elastomeric material is permanently adhered to the lower surface, or upper surface or both upper and lower surfaces; wherein the blade body is substantially arcuate with the convex upper face and a concave lower face; wherein the horizontal portion of the flange is substantially arcuate with the convex upper surface and a concave lower surface; a mounting portion; and one or more light sources; wherein the light source is selectively disposed on or in the vertical portion or the horizontal portion of the flange, the blade body, the distal end or the proximal end of the blade assembly, the distal tip, the mounting portion, the handle, or the edges of the blade assembly. In another aspect, the elastomeric material is permanently adhered circumferentially around the blade. In another aspect, the elastomeric material is adhered circumferentially around the blade, excluding the distal end, the proximal end or the distal and proximal end, and/or around or on the light source(s).

### BRIEF DESCRIPTION OF THE DRAWINGS

These and other features, aspects, and advantages of the present invention will become apparent from the following description, appended claims, and the accompanying exemplary embodiments shown in the drawings, which are briefly described below.

FIG. 1 is a side bottom view of a laryngoscope blade assembly comprising an elastomeric material, wherein the elastomeric material is permanently adhered to a selected part of a surface on a flange of the blade assembly according to one exemplary embodiment.

FIG. 2 is a side top view of a laryngoscope blade assembly comprising an elastomeric material, wherein the elastomeric material is permanently adhered to a selected part of a face of the blade body and to a selected part of the horizontal portion of the flange according to one exemplary embodiment.

FIG. 3 is a cross-sectional view of a laryngoscope blade assembly comprising an elastomeric material, wherein the elastomeric material is permanently adhered to a selected part of the horizontal portion according to one exemplary embodiment.

FIG. 4 is a cross-sectional view of a laryngoscope blade assembly comprising an elastomeric material, wherein the elastomeric material is permanently adhered to the horizontal portion and the lateral side wall of the vertical portion according to one exemplary embodiment.

FIG. 5 is a cross-sectional view of a laryngoscope blade assembly comprising an elastomeric material, wherein the elastomeric material is permanently adhered to the horizontal portion, as observed from the proximal end toward the distal end of the blade body according to one exemplary embodiment.

FIG. 6 is a cross-sectional view of a laryngoscope blade assembly comprising an elastomeric material, wherein the elastomeric material is permanently adhered to the horizontal portion and the medial side wall of the vertical portion according to one exemplary embodiment.

FIG. 7 is a side top view of a laryngoscope blade assembly comprising a handle portion, one or more light sources and an elastomeric material according to one exemplary embodiment.

While the figures illustrate some embodiments of the invention, a person skilled in the art will recognize that the full scope of the invention is not limited by these figures.

### DETAILED DESCRIPTION OF THE EMBODIMENTS

The invention is described in more detail hereinafter with reference to exemplary embodiments. In the figures, the same reference numerals are used for similar components in different embodiments. It is understood that the invention is not limited in its application to the details of construction and the arrangement of components set forth in the following description or illustrated in the following drawings. The invention is capable of other embodiments and of being practiced or of being carried out in various ways. Also, it is understood that the phraseology and terminology used herein is for the purpose of description and should not be regarded as limiting.

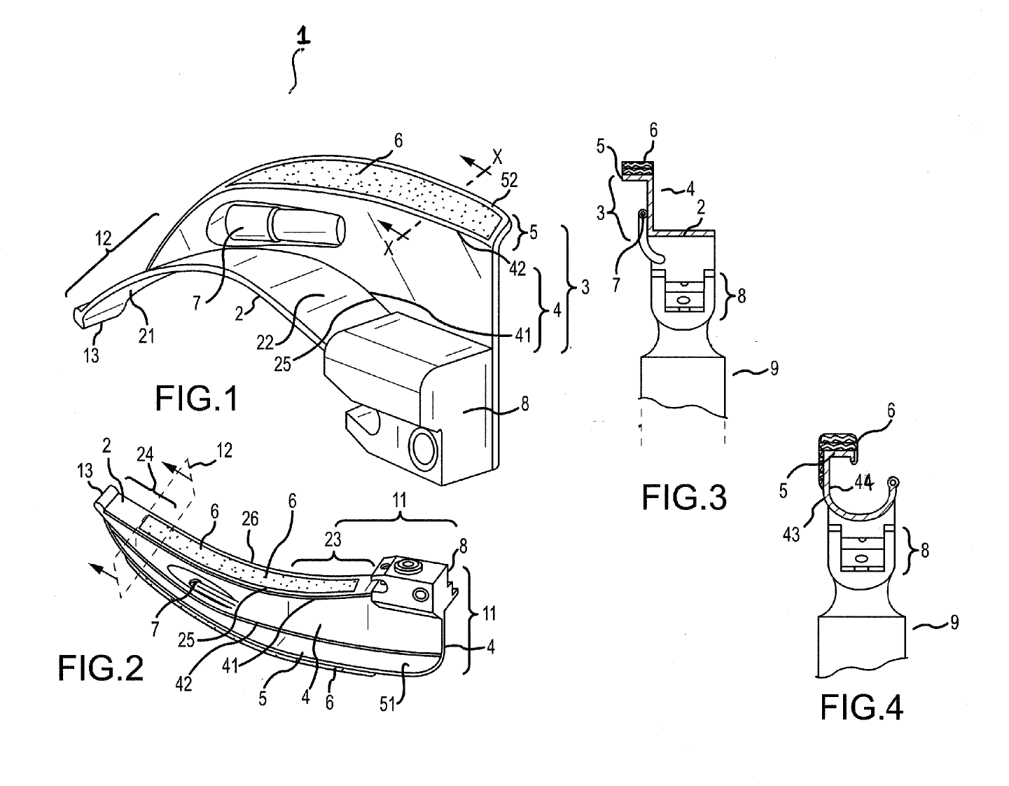

Referring to FIG. 1, according to one exemplary embodiment of the present application, a side bottom view shows a laryngoscope blade assembly 1 comprising a blade body 2 comprising an upper face 21 and a lower face 22, a proximal end 23 and a distal end 24, the upper face 21 and lower face 22 defined by a first edge 25 and a second edge 26, wherein the first edge 25 of the blade body 2 is configured to connect to a top edge 41 of a vertical portion 4 of a flange 3, wherein the flange 3 comprises the vertical portion 4 comprising the top edge 41 and a bottom edge 42, a lateral side wall 43 and a medial side wall 44, the wall defined by the top edge 41 and the bottom edge 42, and the flange 3 further comprising a substantially horizontal portion 5 with an upper surface 51 and a lower surface 52, and an elastomeric material 6 configured on the lower surface 52, wherein the elastomeric material 6 is permanently adhered to the lower surface 52. In one embodiment, the elastomeric material 6 is permanently adhered to the upper surface 51. In another embodiment, the elastomeric material 6 is permanently adhered to both the upper surface 51 and lower surface 52. In another embodiment, the blade assembly 1 further comprises a mounting portion 8.

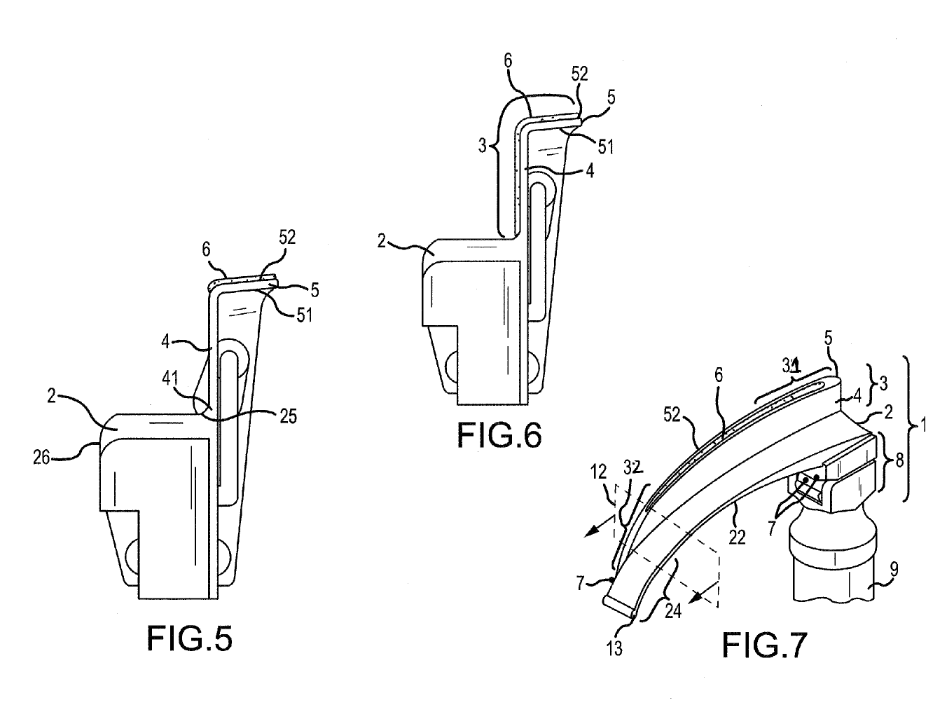

Referring to FIGS. 3, 4, 5 and 6, according to one exemplary embodiment of the present application, a cross-sectional view shows a blade assembly 1 comprising a blade body 2 with upper face 21, a vertical portion 4 with a medial side wall 42 and a lateral side wall 43, a horizontal portion 5 with lower surface 52, an elastomeric material 6 and one or more light sources 7. In one embodiment, the blade assembly 1 further comprises a mounting portion 8 and a handle portion 9. In another embodiment, the elastomeric material 6 is permanently adhered to the upper face 21 and the lower surface 52. In one embodiment, the elastomeric material 6 is permanently adhered to the lateral side wall 43. In another embodiment, the elastomeric material 6 is permanently adhered to a selected part of the lower surface 52, the upper face 21, or the lateral side wall 43. In one embodiment, the elastomeric material 6 is permanently adhered to cover the distal tip 13. In another embodiment, the elastomeric material 6 is permanently adhered to cover distal portion 12 but not the distal tip 13.

Referring to FIG. 2, according to one exemplary embodiment of the present application, a side top view of a blade assembly 1 comprising a blade body 2, a vertical portion 4, a horizontal portion 5, one or more light sources 7 and an elastomeric material 6 is shown. In one embodiment, the elastomeric material 6 is permanently configured on an upper face 21 of the blade body 2 and a lower surface 52 of the horizontal portion 5. In another embodiment, the elastomeric material 6 is permanently adhered to the entire upper face 21 of the blade body 2 and the entire lower surface 52 of the horizontal portion 5. In another embodiment, the elastomeric material 6 is permanently adhered to a selected part of the upper face 21 of the blade body 2 and the lower surface 52 of the horizontal portion 5.

Referring to FIG. 7, according to one exemplary embodiment of the present application, a side bottom view of a blade assembly 1 comprising a handle portion 9, a mounting portion 8, one or more light sources 7, an elastomeric material 6 and lower surface 52 of a horizontal portion 5. In one embodiment, the light source 7 is configured on the mounting portion 8 of the blade assembly 1. In another embodiment, the light source 7 is configured on the blade assembly 1 or the handle portion 9. In one embodiment, the flange 3 comprises a proximal end 31 and a distal end 32.

In one aspect of the present application, there is provided a laryngoscope blade assembly 1 having a proximal end 11, a distal end 12 and a distal tip 13, comprising a blade body 2 comprising an upper face 21 and a lower face 22, a proximal end 23 and a distal end 24, the upper face 21 and lower face 22 defined by a first edge 25 and a second edge 26; wherein the first edge 25 of the blade body 2 is configured to connect to a top edge 41 of a substantially vertical portion 4 of a flange 3 having a proximal end 31 and a distal end 32, wherein the flange 3 comprises the vertical portion 4 comprising the top edge 41 and a bottom edge 42, a lateral side wall 43 and a medial side wall 44, the wall defined by the top edge 41 and the bottom edge 42, wherein the vertical portion 4 extends downward from the first edge 25 of the blade body 2, and the flange 3 further comprising a substantially horizontal portion 5, wherein the horizontal portion 5 comprises an upper surface 51 and a lower surface 52; wherein the horizontal portion 5 is configured to extend laterally or medially from the bottom edge 42 of the vertical portion 4; and an elastomeric material 6 configured on the lower surface 52 of the horizontal portion 5 of the flange 3, wherein the elastomeric material 6 is permanently adhered to the lower surface 52 (or upper surface 51, or both lower and upper surfaces); wherein the blade body 2 is substantially arcuate with the convex upper face 21 and a concave lower face 22; and wherein the horizontal portion 5 of the flange 3 is substantially arcuate with the convex upper surface 51 and a concave lower surface 52.

In one aspect of the present application, the horizontal portion 5 of the flange 3 may be configured to extend medially from the bottom edge 42 of the vertical portion 4, in substantially the same direction as the blade body 2. In one aspect, the horizontal portion 5 is configured to extend laterally from the bottom edge 42 of the vertical portion 4, in a direction substantially away from the blade body 2. In another aspect of the present application, the horizontal portion 5 may be configured to extend from the bottom edge 42 of the vertical portion 4 at an angle that may be substantially perpendicular to the vertical portion 4.

In one aspect, the horizontal portion 5 may be arcuate, wherein the horizontal portion 5 may be configured to curve upward and toward the blade body 2, wherein the upper surface 51 of the horizontal portion 5 may be convex and the lower surface 52 of the horizontal portion 5 may be concave, wherein the distal end 32 of the flange 3 and the distal end 24 of the blade body 2 may intersect.

In another aspect, the horizontal portion 5 may be configured to extend from the bottom edge 42 of the vertical portion 4 wherein the horizontal portion 5 and vertical portion 4 may form a curvature. In one embodiment, the curvature may be substantially medial, wherein the curvature may extend in substantially the same direction as the blade body 2. In another embodiment, the curvature may be substantially lateral, wherein the curvature may extend in substantially the opposite direction from the blade body 2.

In yet another aspect of the present application, the surface of the horizontal portion 5 of the flange 3 may be configured with variable widths. In one embodiment, the width closer to the distal end 32 of the flange 3 may be narrower than the width closer to the proximal end 31. In one aspect of the present application, the distal end 24 and the proximal end 23 of the blade body 2 may be configured with variable widths. In one embodiment, the distal end 24 may have a wider width than the proximal end 23 wherein the two sides of the blade body 2 may distally extend divergently.

In one aspect of the present application, the vertical portion 4 of the flange 3 may be configured to extend from the first edge 25 of the blade body 2 at an angle that may be substantially perpendicular to the lower face 22 of the blade body 2. In another aspect, the vertical portion 4 may be configured to extend from the first edge 25 of the blade body 2 wherein the blade body 2 and the vertical portion 4 may form a curvature.

In one aspect of the present application, the vertical portion 4 and the horizontal portion 5 of the flange 3 may be configured to have the same length as the length of the blade body 2. In another aspect, the vertical portion 4 and the horizontal portion 5 of the flange 3 may be configured to have variable lengths with respect to each other and with respect to the length of the blade body 2. In one embodiment, the vertical member of the flange 3 and the blade body 2 may be configured to have the same length and the horizontal member of the flange 3 may be configured to have a length that may be from about ⅓ to about ¾ or about ½ of the length of the vertical member and the blade body 2. In another embodiment, the horizontal portion 5 of the flange 3 may be configured to have a length that may be from about ⅓ cm to about 2 cm, or from about ½ cm to about 1.5 cm shorter than the length of the vertical portion 4 and the blade body 2. In one embodiment, the vertical portion 4 may have a thickness of 1 mm. In another embodiment, the vertical portion 4 may have a thickness of about 0.9 mm. In one embodiment, the vertical portion 4 may have a width of 1 cm at the proximal end 11 of the blade assembly 1, as measured from the top edge 41 to the bottom edge 42. In one embodiment, the vertical portion 4 may have a width of 0.9 cm at the proximal end 11. In another embodiment, the vertical portion 4 may have a width of 0.5 cm at the distal end 12.

In another aspect, the blade body 2 may have a width of 1.5 cm. In one embodiment, the width may be 1.3 cm or 1.4 cm.

In one aspect, the thickness of the blade body, the vertical portion, or the horizontal portion may be 0.1 cm. In one embodiment, the thickness may be 0.09 cm or 0.2 cm.

In another aspect, the distal tip 13 may be 1.2 cm or 1.3 cm wide.

In another aspect, the horizontal portion 5 may be 1.8 cm wide at the proximal end 11. In one embodiment, the horizontal portion 5 may be 1.5 cm wide at the portion in between the proximal end 11 and the distal end 12. In one embodiment, the horizontal portion 5 may be 0.5 cm at the distal end 12.

In one aspect, the vertical portion 4 of the blade assembly 1 may be 0.5 cm wide.

In another aspect, the horizontal portion 5 may be 0.5 cm wide.

In yet another aspect, the blade body 2 may be 10 cm long. In one embodiment, the blade body 2 may be 13 cm or 16 cm long.

In one aspect, the blade assembly 1 may have size 0, 1, 2, or 3. In one embodiment, the blade assembly 1 may be configured with size appropriate for premature babies, neonatal patients, or children from about 2 to about 8 years old.

In another aspect, the blade assembly 1 may be configured to be used in animal patients. In one embodiment, the blade assembly 1 may be configured to be used in a 5-inch mouth of an equine patient. In another embodiment, the blade assembly 1 may be configured to be used in animals such as dogs, cats, tamed wild animals such as lions, tigers, or avian patients.

In one aspect, the elastomeric material 6 is permanently bonded or fused on the blade body 2 or the flange 3. In another aspect, the elastomeric material 6 permanently configured on both surfaces of the horizontal portion 5 of the flange 3 and the lateral side wall 43 of the vertical portion 4 provides unexpected and non-obvious improvement in displacing and securing the patient's tongue away for a more efficient and rapid endotracheal intubation. In one aspect, the elastomeric material 6 permanently configured on the flange 3 and blade body 2 contacts and retains a patient's tongue during a procedure. In one embodiment, the material prevents slippage or inadvertent movement of the tongue during intubation. In another embodiment, the material securely displaces the tongue to facilitate intubation. In another embodiment, the material prevents slippage or inadvertent movement of the blade inside the mouth or airway of the patient during intubation.

In one aspect of the present application, the elastomeric material 6 may be permanently adhered to a selected part of the lower surface 52 of the horizontal portion 5 of the flange 3. In one embodiment, the elastomeric material 6 may be configured to be permanently adhered to substantially the entire lower surface 52 and the entire upper surface 51 of the horizontal portion 5 of the flange 3. In another embodiment, the material may be permanently adhered to a part of the horizontal portion 5, wherein the part may contact the maxillary teeth, jaw, or gums of the patient. In one embodiment, the part of the horizontal portion 5 may come into contact with a patient's tongue.

In another aspect of the present application, the elastomeric material 6 may be permanently adhered to a selected part of the lateral side wall 43 of the vertical portion 4 of the flange 3. In one embodiment, the elastomeric material 6 may be configured to be adhered to substantially the entire lateral side wall 43 of the vertical portion 4, wherein the material is further configured to adapt one or more light sources 7.

In one aspect, the elastomeric material 6 may comprise an exterior surface and an interior surface, wherein the interior surface is configured to contact and permanently adhere to the blade assembly 1, wherein the exterior surface comprises a textured surface.

In one aspect, the elastomeric material 6 may be configured to be selectively positioned on the lower surface 52 of the horizontal portion 5, wherein the material is permanently adhered to the surface, wherein the material may be permanently adhered to a selected part of the lower surface 52, wherein the material may make contact with the maxillary teeth, jaw, or gums of the patient. In various embodiments, the patient's teeth comprise incisor, canine, pre-molar, or molar teeth. In another aspect of the present application, the elastomeric material 6 permanently adhered to the blade assembly 1 significantly prevents inadvertent displacement or slippage of the tongue when the tongue is secured by the laryngoscope blade assembly 1.

In one aspect, the blade body 2, the flange 3 may be substantially rigid, and may be constructed of a metal or metal alloy, plastic or plastic composite, silicon or silicon composite.

In one aspect, the elastomeric material 6 is configured on the upper face 21 of the blade body 2, wherein the material is permanently adhered to the upper face 21. In one embodiment, the elastomeric material 6 may be configured to be selectively positioned on the upper face 21 of the blade body 2. In another embodiment, the elastomeric material 6 may be configured on the upper face 21, wherein the material may make contact with the mandibular teeth, jaw, or gums of the patient. In another aspect, the elastomeric material 6 may be configured to be permanently adhered to substantially the entire upper face 21 of the blade body 2. In one aspect, the elastomeric material 6 may be permanently adhered to a selected part of the upper face 21 of the blade body 2. In one embodiment, the material 6 may be permanently adhered to the distal end 24 or the proximal end 23 of the blade body 2, wherein the material 6 is permanently adhered to the upper face 21 of the blade body 2. In another embodiment, the material 6 may be permanently adhered to a selected part of the upper face 21, wherein the material 6 adhered to the part that may come into contact with the mandibular teeth, jaw, or gums of the patient.

In all of the foregoing aspects of the present application, there is provided a laryngoscope blade assembly 1, wherein the elastomeric material 6 may be configured on the upper face 21 or a selected part of the upper face 21 of the blade body 2, on the lower surface 52 or a selected part of the lower surface 52 of the flange 3, wherein the material 6 is permanently adhered to the surface and the face, wherein the material 6 may be selectively positioned such that the material 6 may make contact with the patient's upper or lower teeth or jaws when the laryngoscope blade assembly 1 is inserted into the patient's mouth.

In other aspects of the present application, the elastomeric material 6 may be selectively positioned at any point along the blade body 2 or the flange 3, wherein the material 6 is permanently adhered to the blade body 2 or the flange 3. In one embodiment, the material 6 may be configured on the distal end 24 of the blade body 2. In one embodiment, the distal end 24 configured with the material 6 prevents inadvertent or abrupt slippage of the blade assembly 1 when the blade assembly 1 is rested on the wet surface inside a patient's mouth. As a result, the distal end 24 configured with the material 6 prevents injury or tear to the oral mucosa, the epiglottis, or the airway of the patient.

In one aspect, the material 6 may be configured to cover the distal end 24 of the blade body 2 from about 0.5 cm to about 5 cm, from about 1.5 cm to about 4 cm, or from about 2 cm to about 3 cm from the distal tip 13 of the blade assembly. In another embodiment, the material 6 may be configured to cover the distal end 32 of the flange 3. In yet another embodiment, the material 6 may be configured from about 0.5 cm to about 5 cm, from about 1.5 cm to about 4 cm, or from about 2 cm to about 3 cm from the distal tip 13 of the blade assembly 1.

In another aspect, the elastomeric material 6 is configured on the lateral side wall 43 of the vertical portion 4 of the flange 3, wherein the material 6 is permanently adhered to the lateral side wall 43. In one aspect, the elastomeric material 6 comprises one or more layers. In one embodiment, the elastomeric material 6 may comprise at least 1, 2, 3, 4, or more layers. In another aspect, the elastomeric material 6 may comprise one or more layers, wherein the layers may comprise the same elastomeric material 6. In one embodiment, the layers may comprise different elastomeric materials. In another embodiment, the materials may be selected from a group consisting of silicone, silicone composite, rubber, latex, and other elastomeric materials.

In yet another aspect of the present application, the elastomeric material 6 may be reversibly and compressibly deformable. In one embodiment, upon release from contact with the patient's teeth, gums, or jaw, the elastomeric material 6 may return to its original, pre-contact shape. In one aspect, the elastomeric material may be coated with an antibacterial or antiseptic material as known in the art.

In all of the foregoing aspects, the elastomeric material 6 may be permanently adhered to the blade assembly 1, wherein the material 6 is non-detachable, non-slidable and non-movable from its position configured on the blade assembly 1.

In one aspect, the elastomeric material 6 may be circumferentially configured on the blade assembly 1, wherein the material may be permanently adhered to the upper face 21, the first edge 25 and the second edge 26 of the blade body 2, the lateral side wall 43 of the vertical portion 4 of the flange 3, the upper surface 51 and the lower surface 52 of the horizontal member 5 of the flange 3.

In one aspect, the elastomeric material 6 is adaptively configured to enclose one or more air-tight cavities, wherein the cavity is adapted to enclose an air pocket. In one embodiment, the elastomeric material 6 configured with one or more air pockets tactilely provides an improved feedback to the user that the laryngoscope blade body 2 is about to collide with blunt force with the patient's teeth, gums, or jaw if the blade is pushed or lifted further. In another embodiment, the material 6 provides additional early warning when the blade assembly 1 is inadvertently rocked using the patient's upper teeth, gums, or jaw as a fulcrum.

In one aspect wherein the elastomeric material 6 is adaptively configured to incorporate one or more light sources 7, wherein the light source 7 is configured on the vertical portion 4 of the flange 3, the horizontal portion 5 of the flange 3, the blade body 2, the distal end 12 and proximal end 11 of the blade assembly 1, the distal tip 13 of the blade assembly 1, and combinations thereof. In one embodiment, the light source 7 may be configured to be completely or partially disposed within the vertical portion 4 of the flange 3. In another embodiment, the light source 7 may be configured to be disposed within the blade body 2. In yet another embodiment, the light source 7 may be disposed on the horizontal portion 5 of the flange 3.

In another aspect, the light sources 7 may comprise one or more lights, wherein the light source may be disposed within the vertical and horizontal portion 5 of the flange 3 and the blade body 2, wherein in the light sources may be disposed on or near the distal end 32 of the flange 3 and the distal end 24 of the blade body 2. In one aspect, the light source 7 may be disposed directionally longitudinally along the blade body 2. In various embodiments, the light source 7 may be selectively positioned or disposed on the blade body 2. In another aspect, the light source 7 may comprise one or more lights configured in a ring formation, wherein the ring formation of lights may be configured circumferentially on the blade assembly 1. In one aspect, one or more lights may be configured as a bank of lights, wherein the light may be configured on the distal portion of the blade assembly 1. In one embodiment, the light source 7 may comprise a bank of lights, wherein the bank of lights may be configured on the proximal end 11 of the blade assembly 1. In another aspect, the light source 7 may comprise one or more banks of lights, or one or more rings of lights, wherein the banks or rings of lights may be selectively positioned or configured on the blade assembly 1.

In one aspect, one or more lights may be configured to be disposed at a predetermined distance from each other, wherein the lights may be configured to be selectively positioned or configured on the blade assembly 1. In another aspect, the light source 7 may comprise one or more high-intensity discharge miniature lights. In one embodiment, the light may be LED or Xenon light. In various embodiments, the light may be replaceable. In one embodiment, the light source 7 may be configured to be a continuous band of light. In one aspect, there is provided a laryngoscope blade assembly 1 comprising an electrical conduit or continuity configured to be attached to one or more light sources 7.

In all of the foregoing aspects, the elastomeric material 6 may be configured to be permanently adhered to the light source 7 and the blade assembly 1, wherein the material 6 may be adaptively configured to expose only the light bulb, wherein the light bulb may provide sufficient light and visibility within the mouth and airway of the patient.

In one aspect, the elastomeric material 6 comprises an exterior surface and an interior surface, wherein the interior surface is configured to contact and permanently adhered to the blade assembly 1, wherein the exterior surface comprises a textured surface selected from bumps, raised protrusions, knobs, riblets, waves, ridges, ribs, raised truncated domes or half domes and combinations thereof. In one embodiment, the textured surface may comprise corrugated texture.

When the laryngoscope is placed in a patient's mouth and the textured surface of the blade is placed in contact with the oral mucosa, the textured surface provides a static, frictional or gripping surface with the oral mucosa to prevent movement or slippage of the blade during use. In addition, when the tongue is displaced and securely engaged and in contact with the blade surface, the textured surface provides a static, frictional or gripping surface with the tongue to secure the tongue, preventing movement or slippage of the tongue. In one aspect, the textured surface may provide the laryngoscope blade assembly 1 with an improved captivatable contact, arrestable retention, and statically securing of the tongue when the laryngoscope blade assembly 1 is inserted into the patient's mouth or airway.

In one aspect, the vertical portion 4 and the horizontal portion 5 of the flange 3 may be adaptively configured to have variable width and height to captivatably contact and arrestably secure the patient's tongue. Since the size of the tongue varies from patient to patient, the user may choose the blade assembly 1 with the appropriate flange and blade body to effectively displace the tongue during laryngoscopy.

In another aspect, the textured surface may allow the user to wash or clean the elastomeric material 6 and the laryngoscope blade assembly 1 quickly and efficiently. In one embodiment, the textured surface allows oral residues, secretions, fluid, and matters from the patient to easily dislodge from between the knobs, waves, ribs, riblets, bumps, raised protrusions raised truncated domes or half domes, or ridges. In one aspect, the textured surface of the elastomeric material 6 may prevent cross-contamination.

In one aspect, the elastomeric material 6 and the lower surface 52 of the horizontal portion 5 of flange 3 comprise a substantially continuous surface. In another aspect, the elastomeric material 6 and the upper face 21 of the blade body 2 comprise a substantially continuous surface.

In one aspect, the elastomeric material 6 and the lateral side wall 43 of the vertical portion 4 of the flange 3 comprise a substantially continuous surface. In one embodiment, the substantially continuous surface may be configured to be permanently sealed, wherein the surface may be air-tight and liquid tight, wherein no contaminated liquid matter or air can enter the space sealably disposed between the elastomeric material 6 and the blade assembly 1. In another aspect, the elastomeric material 6 and the upper face 21 of the blade body 2 or the lower surface 52 of the horizontal portion 5 of the flange 3 may form a continuous curved surface.

In one aspect, the blade is selected from the group consisting of a Macintosh, Miller and Magill blade. In another aspect, the blade is selected from the group consisting of Phillips, Robertshaw, Sykes, Wisconsin, and Wis-Hipple blade. In another embodiment, the blade may be curved, partially curved or straight, with or without mirror or light source, with or without curved tip.

In one aspect, the blade body 2 and the flange 3 may be formed from one single piece of material by methods such as stamping or bending to first form the blade body 2, then the vertical portion 4, then the horizontal portion 5. In one embodiment, the single piece of material may be metal or metal alloy, or plastic. In another embodiment, the single piece of material may be rigid materials. In one embodiment, the blade body 2 and the flange 3 may be formed from stainless steel or titanium or aluminum.

In one aspect of the present application, there is provided a process of configuring an elastomeric material 6 on the lower surface 52 of the horizontal portion 5 of the flange 3 of a laryngoscope blade assembly 1, the process comprises fusing, bonding, gluing, baking, heat-curable adhesion, form-in-place coating, thermo-setting or heat curing, and pressurizing. In another aspect, the process comprises preparing a selected part of the lower surface 52 of the flange 3 and permanently adhering the elastomeric material 6 to the selected part. In one aspect, the process comprises preparing the upper surface 51 of the horizontal portion 5 of the flange 3 or a selected part of the upper surface 51. In another aspect, the process comprises preparing the lateral wall of the vertical portion 4 of the flange 3, and the upper face 21 and the lower face 22 of the blade body 2, or a selected part thereof. In one aspect, the method comprises preparing the surface, face, or edge of the blade assembly 1 and applying the elastomeric material 6 to the surface, face or edge under conditions appropriate for permanently adhering the elastomeric material 6 to a metal or metal alloy surface. In one embodiment, the process comprises pressure and heat. In another aspect, the process comprises high grade elastomeric material, wherein the material may be impervious to foreign object damage, resistant to erosion and safe for sterilizing procedures and conditions.

In one aspect, the elastomeric material 6 comprises a thickness from about 1 mm to about 10 mm, from about 2 mm to about 10 mm, from about 4 mm to about 8 mm, and from about 5 mm to about 7 mm. In one embodiment, the elastomeric material 6 comprises a thickness from about 1 mm to about 10 mm, from about 2 mm to 20 mm, from about 5 mm to about 15 mm, from about 10 mm to about 12 mm. In one embodiment, the elastomeric material 6 may be 1 mm, 1.5 mm, 2 mm, or 2.5 mm.

In one aspect of the present application, there is provided a laryngoscope blade assembly 1 having a proximal end 11, a distal end 12 and a distal tip 13, comprising a blade body 2 comprising an upper face 21 and a lower face 22, a proximal end 23 and a distal end 24, the upper face 21 and lower face 22 defined by a first edge 25 and a second edge 26; wherein the first edge 25 of the blade body 2 is configured to connect to a top edge 41 of a substantially vertical portion 4 of a flange 3 having a proximal end 31 and a distal end 32, wherein the flange 3 comprises the vertical portion 4 comprising the top edge 41 and a bottom edge 42, a lateral side wall 43 and a medial side wall 44, the wall defined by the top edge 41 and the bottom edge 42, wherein the vertical portion 4 extends downward from the first edge 25 of the blade body 2, and the flange 3 further comprising a substantially horizontal portion 5, wherein the horizontal portion 5 comprises an upper surface 51 and a lower surface 52; wherein the horizontal portion 5 is configured to extend medially from the bottom edge 42 of the vertical portion 4, in substantially the same direction as the blade body 2; and an elastomeric material 6 configured on the lower surface 52 of the horizontal portion 5 of the flange 3, wherein the elastomeric material 6 is permanently adhered to the lower surface 52; wherein the blade body 2 is substantially arcuate with the convex upper face 21 and a concave lower face 22; and wherein the horizontal portion 5 of the flange 3 is substantially arcuate with the convex upper surface 51 and a concave lower surface 52.

In various aspects of the above, the laryngoscope blade assembly 1 further comprises a mounting portion 8 and a handle 9, wherein the handle 9 is pivotally and lockably attached to the mounting portion 8 of the laryngoscope blade assembly 1. In one embodiment, the handle 9 may be removably attached to the mounting portion 8. In another embodiment, the handle 9 may be permanently attached to the mounting portion 8. In one aspect, the handle 9 is adaptively configured with a power source and electrical continuity to one or more light sources 7 configured on the blade assembly 1.

In one aspect, the handle 9 is adaptively configured with an internal light source 7 and an optical conduit, wherein the internal light source 7 is disposed within the handle 9. In one embodiment, the optical conduit may be an optical fiber having a first and second ends, the first end may be attached to the internal light source 7, wherein the optical fiber may provide an optical continuity, wherein light from the internal light source 7 may be transmitted inside the optical fiber and may exit the optical fiber on the second end. In one embodiment, the second end of the optical fiber may be selectively disposed on or in the flange 3 or the blade body 2. In another embodiment, the second end may be selectively disposed on the distal portion of the blade assembly 1.

In one aspect of the present application, there is provided a laryngoscope blade assembly 1 having a proximal end 11, a distal end 12 and a distal tip 13, comprising a blade body 2 comprising an upper face 21 and a lower face 22, a proximal end 23 and a distal end, the upper face 21 and lower face 22 defined by a first edge 25 and a second edge 26; wherein the first edge 25 of the blade body 2 is configured to connect to a top edge 41 of a substantially vertical portion 4 of a flange 3 having a proximal end 31 and a distal end 32, wherein the flange 3 comprises the vertical portion 4 comprising the top edge 41 and a bottom edge 42, a lateral side wall 43 and a medial side wall 44, the wall defined by the top edge 41 and the bottom edge 42, wherein the vertical portion 4 extends downward from the first edge 25 of the blade body 2, and the flange 3 further comprising a substantially horizontal portion 5, wherein the horizontal portion 5 comprises an upper surface 51 and a lower surface 52; wherein the horizontal portion 5 is configured to extend medially from the bottom edge 42 of the vertical portion 4; and an elastomeric material 6 configured on the lower surface 52 of the horizontal portion 5 of the flange 3, wherein the elastomeric material 6 is permanently adhered to the lower surface 52; wherein the blade body 2 is substantially arcuate with the convex upper face 21 and a concave lower face 22; wherein the horizontal portion 5 of the flange 3 is substantially arcuate with the convex upper surface 51 and a concave lower surface 52; a mounting portion 8; and one or more light sources 7; wherein the light source 7 is selectively disposed on or in the vertical portion 4 or the horizontal portion 5 of the flange 3, the blade body 2, the distal end 12 or the proximal end 11 of the blade assembly 1, the distal tip 13, the mounting portion 8, the handle 9, or the edges of the blade assembly 1.

In another aspect of the application, there is provided a laryngoscope blade assembly 1 according to Table 1, wherein the blade assembly 1 comprises the types of blade and any ancillary feature according to Table 1. In one embodiment, the blade assembly 1 comprises a curved Macintosh blade. In another embodiment, the blade assembly 1 comprises a curved Macintosh blade with a reduced flange. In another embodiment, the blade assembly 1 comprises a Miller blade, a flange 3 or a reduced flange, an integrated mirror, a light source 7, and light-carrying optical fibers. In yet another embodiment, the blade assembly 1 comprises a Magill blade, wherein the blade comprises a curved or a straight blade, a single- or multiple-light source 7, optionally a flange 3 or a reduced flange, optionally a mirror, and optionally light-carrying optical fibers.

TABLE 1

| TYPE of BLADE | | ANCILLARY FEATURES |
|---|---|---|
| Reduced Flange | Macintosh | Curved or Straight |
| Cranwall | Magill | Flange or Reduced Flange |
| Jackson | Miller | Integrated mirror |
| Wis-Hipple | Wisconsin | Light ring comprising one or more |
| Robertshaw | Phillips | miniature lights |
| Siker | Parrott | Single light source, bank of lights |
| Soper | Janeway | Fiber optics |
| | | Light-carrying optical fibers |

In one aspect of the present application, there is provided a laryngoscope blade assembly 1, wherein the blade body 2 may comprise a curved or straight shape, a flange 3, or an integrated mirror. In various embodiments of the above, the blade body 2 may comprise any combination of blade and additional ancillary features according to Table 1. In one embodiment, the blade body 2 may comprise a curved Macintosh blade, a light source 7, and a light ring. In yet another embodiment, the blade body 2 may comprise a straight Miller blade having a curved distal tip 13, a light ring and optionally an integrated mirror.

In one aspect of the present application, there is provided a process of configuring an elastomeric material 6 on the lower surface 52 of the horizontal portion 5 of the flange 3 of the laryngoscope blade assembly 1, wherein the elastomeric material 6 is permanently adhered to the lower surface 52, comprising manufacturing the elastomeric material 6, wherein the material 6 is configured to enclose one or more cavities, wherein the cavity is adaptively configured to enclose an air pocket, preparing the lower surface 52 to receive the elastomeric material 6, and permanently adhering the elastomeric material 6 to the lower surface 52.

Although the foregoing embodiments of the present application have been described by way of examples and illustrations, one skilled in the art will recognize and appreciate that many modifications and adaptations can be made to the various aspects of the invention described herein and can even be desirable, while still being within scope of the present invention. One skilled in the art will also recognize that some features of the present invention may be used without utilizing other features to achieve some desired results. Therefore, the above embodiments illustrate some of the aspects of the present invention and are not in limitation thereof.

### DEFINITIONS

The term “about” as used herein may refer to ranges of value expressed in “from about” a value “to about” a value.

The term “alveolar process” as used herein refers to the thickened ridge of bone or raised thickened border on each side of the upper or lower jaw that contains the sockets of the teeth.

The term “blade” or “laryngoscope blade” as used herein refers to commonly used blade, such as a metal or plastic blade, for examination of a patient's airway. Accordingly, the blade may be a Macintosh, Miller, Cranwall, Janeway, Magill, or any other type of laryngoscope blade. In one aspect, the blade comprises a proximal and a distal end, wherein the proximal end is the portion of the blade that is closer to the handle of the laryngoscope and the distal end is the portion of the blade that is further away from the handle of the laryngoscope.

The term “blade assembly” refers to a blade or a laryngoscope blade having a flange and an elastomeric material, further comprising a light or light source, electrical or optical conduit, and base or mounting portion for connecting or contacting with a handle of a laryngoscope. In one aspect, the blade assembly may be configured to be adapted for a left hand user or symmetrically adapted for a right hand user.

The term “cavity” as used herein refers to a hollow space within the body of an object. Accordingly, a cavity enclosed in an elastomeric material may refer to a hollow space entirely enclosed within the elastomeric material. An air-tight cavity may refer to a cavity containing an air pocket, wherein the air cannot escape from the cavity.

The term “composite” as in “silicone composite” as used herein refers to engineered or naturally occurring materials comprising silicone and one or more constituent materials. Silicone composite may comprise fillers. Silicone composite is generally strong and flexible, non-reactive, water-proof, stable and resistant to extreme conditions and temperatures.

The term “concave” as used herein refers to curving inward or down or hollowed inward or downward wherein the object is curved like the inner surface of a sphere.

The term “convex” as used herein refers to curving out or up or bulging outward or upward wherein the object is curved like the outer surface of a sphere.

The term “continuous” or “continuous surface” as used herein refers to a substantially continuous transition from the exterior surface of the elastomeric material to the surface and side wall of the flange, the face of the blade body, or the edges of the blade assembly. Accordingly, the elastomeric material may be configured on the blade assembly, wherein the material may form a substantially continuous surface with the blade, wherein the exterior surface of the material may or may not match the shape of the blade body.

The term “curved” or “curvature” as used herein refers to a shape that is formed into a curve. In one embodiment, the distal portion of the blade body may be curved directionally longitudinally. In other embodiments, the edges of the blade body may be curved upward or downward. Accordingly, curvature may refer to the degree of being curved.

The term “elasticity” or “elastic” as used herein refers to the ability of a material such as silicon or rubber to be deformed and to revert to substantially the original form. Elastic deformation is a substantially reversible deformation of a material.

The term “corrugated” as used herein refers to the texture of the exterior surface of the elastomeric material wherein the texture may comprise alternate ridges and grooves.

The term “distal” as used herein refers to the portion of the blade that is further away from the mounting portion of the laryngoscope blade assembly. Accordingly, the distal tip of the blade is the tip furthest away from the mounting portion. The term distal when used in “the distal end of the handle” refers to the end of the handle that is further away from the mounting portion.

The term “elastomers” or “elastomeric” as used herein refers to materials comprising polymers having elasticity and resiliency and may be capable of recovering their original shape after being deformed. The elastomeric material or material may include, for example, rubber, silicone, latex, polyurethane or other aromatic compounds.

The term “electrical conduit” as used herein refers to electrical circuit or routing capable of conducting electrical current.

The term “endotracheal” as used herein refers to being placed within or into the trachea, applied or affected through the trachea. Accordingly, endotracheal intubation refers to a medical procedure in which a tube is placed into the windpipe (trachea) through the mouth.

The term “edge” as used herein refers to the edge of the blade body and of portion of the flange of the blade assembly that when in use may contact the teeth, jaw, gums, or other parts of the airway of the patient. Additionally, the distance between the edges of the blade body or the vertical portion of the flange may define a dimension of the blade body or the vertical portion of the flange.

The term “epiglottis” as used herein refers to a flap of cartilage located in the throat behind the tongue and in front of the larynx.

The term “face” as used herein refers to the face of the blade body that when in use may contact the patient teeth, jaw, gums, oral mucosa, or other parts of the airway. Accordingly, the upper face of the blade body may come into contact with the lower teeth, lower jaw, or lower gums of the patient.

The term “flange” as used herein refers to a part of the laryngoscope blade assembly that may be used to secure the patient's tongue away from the endotracheal tube during laryngoscopy or endotracheal intubation. Accordingly, the flange may be configured perpendicular, at an obtuse, or acute angle to the blade. Additionally, the flange and the blade may be constructed as a unitary construction.

The terms “intubation” and “endotracheal intubation” as used herein may interchangeably refer to the placement of a flexible tube into the trachea (windpipe) to maintain an open airway or to serve as a conduit through which to administer certain drugs. Intubation may be performed in critically injured, ill or anesthetized patients to facilitate ventilation of the lungs, including mechanical ventilation, and to prevent the possibility of asphyxiation or airway obstruction. In one aspect, intubation comprises orotracheal intubation, wherein an endotracheal tube is passed through the mouth and vocal apparatus into the trachea.

The term “knob” or “knobby” as user herein refers to a substantially rounded protrusion from a surface.

The term “laryngoscope” as used herein refers to a medical instrument used in laryngoscopy to examine, obtain a view of the larynx, or to deliver drugs or anesthetic agents into the patient. A laryngoscope may be used to facilitate tracheal intubation during general anesthesia, to perform cardiopulmonary resuscitation, or for procedures on the larynx. In one aspect, laryngoscopy may be performed with the patient lying on his back; the laryngoscope may be inserted into the mouth of the patient, with the distal end of the handle pointing away from the mouth. The user may grasp and pull the handle in the same direction that the distal end of the handle is pointing to.

The term “larynx” or “laryngeal” as used herein refers to the organ in the neck of human or animals including amphibians, mammals, or reptiles involved in breathing, sound production, and protecting the trachea against food aspiration.

The term “lateral” as used herein refers to areas further from, or facing or movement away from the midline.

The term “lower surface” as used herein refers to the surface of the flange that when in used may contact the patient teeth, jaw, gums, oral mucosa, or other parts of the airway. Accordingly, the lower surface of the horizontal portion of the flange may come into contact with the upper teeth, upper jaw, or upper gums of the patient.

The term “mandibular” or “mandible” as used herein refers to the lower jaw, relating to the lower jaw, or associated with the lower jaw in human and veterinarian patients. Accordingly, mandibular injury may refer to injury sustained by the patient's lower jaw.

The term “material” as in “elastomeric material” as used herein refers to the material configured on the laryngoscope blade assembly. In one aspect, the material is configured on the blade body or the flange. In one aspect, the material comprises elastomeric materials or elastomeric materials composite.

The term “maxillary” or “maxilla” as used herein refers to the upper jaw, relating to the upper jaw, or associated with the upper jaw of human or veterinary patients. Accordingly, maxillary injury may refer to injury sustained by the patient's upper jaw.

The term “medial” as used herein refers to areas closer to, facing or movement toward the midline. Accordingly, medial movement may refer to the movement toward an imaginary midline running through an object and dividing the object into equal right and left parts. In one aspect, the imaginary midline runs through the blade body longitudinally, dividing the blade body, and the distal portion and the proximal portion of the blade body into equal right and left parts.

The term “midline” as used herein refers to an imaginary line that may run down the center of the laryngoscope blade substantially parallel to the longitudinal axis of the blade, equidistant from the two edges of the blade, and separating the blade into two imaginary parts, left and right.

The term “unitary construction” as used herein refers to the construction of the laryngoscope blade assembly and the handle as one single piece of equipment without any interlocking and pivotable parts. In one embodiment, the blade assembly and the handle are formed from a single mold to form a unitary piece of equipment.

The term “optical continuity” as used herein refers to the trapping of light from a light source and transmitting the trapped light from one end of and inside an optical conduit without loss of light intensity, wherein the trapped light exits the other end of the optical conduit. The optical conduit may be an optical fiber.

The term “patient” as used herein may refer to human and veterinary patients. In one embodiment, human patient may refer to a human patient without a particular reference to age or sex. In another embodiment, veterinary patients may refer to a veterinary patient without a particular reference to age or sex. Accordingly, patients comprise newborn or adult, human or animals, and male or female.

The term “permanently adhered” as used herein refers to the adherence of the elastomeric material to the blade assembly by any appropriate methods, wherein the material is non-detachable, non-slidable and non-movable from its position on the blade assembly.

The term “proximal” as used herein refers to the end of the laryngoscope blade or the laryngoscope blade assembly that is nearer to the laryngoscope handle. Accordingly, the proximal end of the laryngoscope blade is the end that is closer to the laryngoscope handle. The term may also refer to the proximal end of the laryngoscope handle which is the end closer to the mounting portion.

The term “ribs” or “riblets” as used herein refers to a texture having a pattern of raised bands. Accordingly, a ribbed texture of the exterior surface of the elastomeric material may comprise one or more raised bands.

The term “ridge” or “ridged” as in “ridged texture” as used herein refers to a texture having or forming into or rising up as narrow raised bands.

The term “texture” as used herein refers to the texture of the exterior surface of the elastomeric material that may make contact with the patient's teeth, tongue, gums, jaw, oral mucosa, airway, or epiglottis, wherein the texture may be tactilely perceptible to the user or to the patient.

The term “trachea” as used herein refers to the windpipe which is one part of the airway system.

The term “veterinary” or “veterinarian” as used herein refers to the medical care for the health of animals. Accordingly, a veterinary physician is a doctor who practices veterinary medicine by treating disease, disorder, and injury in non-human animals.

The term “wave” or “wavy” as used herein refers to the texture of the exterior surface of the elastomeric material wherein the texture comprises undulating and wavelike curves.

It is intended that the terminology used herein is only for the purpose of describing some aspects of the invention and is not intended to be limiting in scope.

Although the foregoing embodiments of the present invention have been described in some details by way of illustrations and examples for the limited purposes of clarity and understanding, it will be apparent to those skilled in the art that certain changes and modifications may be practiced within the spirit and scope of the present invention. Other embodiments of the invention will be apparent to those skilled in the art from consideration of the specification disclosed herein. It is intended that the present invention cover the modifications and variations of this invention provided they come within the scope of the appended claims and their equivalents. Therefore, the specification, figures and embodiments presented herein should not be construed to limit the scope of the present invention, but are intended to be considered as exemplary only.

What is claimed is:

1. A laryngoscope blade assembly having a proximal end, a distal end and a distal tip, comprising:
a blade body comprising an upper face and a lower face, a proximal end and a distal end, the upper face and lower face defined by a first edge and a second edge;
wherein the first edge of the blade body is configured to connect to a top edge of a substantially vertical portion of a flange having a proximal end and a distal end, wherein the flange comprises the vertical portion comprising the top edge and a bottom edge, a lateral side wall and a medial side wall, the wall defined by the top edge and the bottom edge, wherein the vertical portion extends downward from the first edge of the blade body, and the flange further comprising a substantially horizontal portion, wherein the horizontal portion comprises an upper surface and a lower surface;
wherein the horizontal portion is configured to extend laterally or medially from the bottom edge of the vertical portion; and
an elastomeric material configured on the lower surface of the horizontal portion of the flange, wherein the elastomeric material is permanently adhered to the lower surface, the upper surface or the lower and the upper surfaces;
wherein the blade body is substantially arcuate with the convex upper face and a concave lower face; and
wherein the horizontal portion of the flange is substantially arcuate with the convex upper surface and a concave lower surface.

2. The laryngoscope blade assembly of claim 1, wherein the horizontal portion is configured to extend laterally from the bottom edge of the vertical portion, in a direction substantially away from the blade body.

3. The laryngoscope blade assembly of claim 2, wherein the elastomeric material is configured on the lateral side wall of the vertical portion of the flange, wherein the material is permanently adhered to the lateral side wall.

4. The laryngoscope blade assembly of claim 3, wherein the elastomeric material comprises one or more layers.

5. The laryngoscope blade assembly of claim 4, wherein the elastomeric material is adaptively configured to enclose one or more air-tight cavities, wherein the cavity is adapted to enclose an air pocket.

6. The laryngoscope blade assembly of claim 5, wherein the elastomeric material is adaptively configured to incorporate one or more light sources, wherein the light source is configured on the vertical portion of the flange, the horizontal portion of the flange, the blade body, the distal end and proximal end of the blade assembly, the distal tip of the blade assembly, and combinations thereof.

7. The laryngoscope blade assembly of claim 6, wherein the elastomeric material comprises an exterior surface and an interior surface, wherein the interior surface is configured to contact and permanently adhere to the blade assembly, wherein the exterior surface comprises a textured surface selected from the group consisting of bumps, raised protrusions, knobs, riblets, waves, ridges, ribs, raised truncated domes or half domes and combinations thereof.

8. The laryngoscope blade assembly of claim 7, wherein the elastomeric material and the lower surface of the horizontal portion of flange comprise a substantially continuous surface.

9. The laryngoscope blade assembly of claim 8, wherein the elastomeric material and the upper face of the blade body comprise a substantially continuous surface.

10. The laryngoscope blade assembly of claim 9, wherein the elastomeric material and the lateral side wall of the vertical portion of the flange comprise a substantially continuous surface.

11. The laryngoscope blade assembly of claim 10, wherein the blade is selected from the group consisting of a Macintosh, Miller or Magill blade.

12. A laryngoscope blade assembly having a proximal end, a distal end and a distal tip, comprising:
a blade body comprising an upper face and a lower face, a proximal end and a distal end, the upper face and lower face defined by a first edge and a second edge;
wherein the first edge of the blade body is configured to connect to a top edge of a substantially vertical portion of a flange having a proximal end and a distal end, wherein the flange comprises the vertical portion comprising the top edge and a bottom edge, a lateral side wall and a medial side wall, the wall defined by the top edge and the bottom edge, wherein the vertical portion extends downward from the first edge of the blade body, and the flange further comprising a substantially horizontal portion, wherein the horizontal portion comprises an upper surface and a lower surface;
wherein the horizontal portion is configured to extend medially from the bottom edge of the vertical portion, in substantially the same direction as the blade body; and
an elastomeric material configured on the lower surface of the horizontal portion of the flange, wherein the elastomeric material is permanently adhered to the lower surface, the upper surface or the lower and the upper surfaces;
wherein the blade body is substantially arcuate with the convex upper face and a concave lower face; and
wherein the horizontal portion of the flange is substantially arcuate with the convex upper surface and a concave lower surface.

13. The laryngoscope blade assembly of claim 12, wherein the elastomeric material comprises a thickness from about 2 mm to about 10 mm, from about 4 mm to about 8 mm, and from about 5 mm to about 7 mm.

14. The laryngoscope blade assembly of claim 13, further comprising a mounting portion and a handle having a proximal end and a distal end, wherein the handle is pivotally and lockably attached at the proximal end to the mounting portion of the laryngoscope blade assembly.

15. The laryngoscope blade assembly of claim 14, wherein the handle is adaptively configured with a power source and electrical conduit to one or more light sources configured on the blade assembly.

16. The laryngoscope blade assembly of claim 15, wherein the handle is adaptively configured with an internal light source and an optical conduit, wherein the internal light source is disposed within the handle.

17. A laryngoscope blade assembly having a proximal end, a distal end and a distal tip, comprising:
a blade body comprising an upper face and a lower face, a proximal end and a distal end, the upper face and lower face defined by a first edge and a second edge;
wherein the first edge of the blade body is configured to connect to a top edge of a substantially vertical portion of a flange having a proximal end and a distal end, wherein the flange comprises the vertical portion comprising the top edge and a bottom edge, a lateral side wall and a medial side wall, the wall defined by the top edge and the bottom edge, wherein the vertical portion extends downward from the first edge of the blade body, and the flange further comprising a substantially horizontal portion, wherein the horizontal portion comprises an upper surface and a lower surface;
wherein the horizontal portion is configured to extend medially from the bottom edge of the vertical portion; and
an elastomeric material configured on the lower surface of the horizontal portion of the flange, wherein the elastomeric material is permanently adhered to the lower surface, the upper surface or the lower and the upper surfaces;
wherein the blade body is substantially arcuate with the convex upper face and a concave lower face;
wherein the horizontal portion of the flange is substantially arcuate with the convex upper surface and a concave lower surface;
a mounting portion; and
one or more light sources;
wherein the light source is selectively disposed on or in the vertical portion or the horizontal portion of the flange, the blade body, the distal end or the proximal end of the blade assembly, the distal tip, the mounting portion, the handle, or the edges of the blade assembly.

18. The laryngoscope blade assembly of claim 13, further comprising a mounting portion and a handle having a proximal end and a distal end, wherein the handle and the mounting portion of the laryngoscope blade assembly are constructed as a unitary construction.

19. The laryngoscope blade assembly of claim 18, wherein the blade assembly is a disposable blade assembly.
